(19) United States
(12) Patent Application Publication
(10) Pub. No.: US 2016/0184515 A1
(43) Pub. Date: Jun. 30, 2016

(54) Electrolytic Diaphragm Pumps Having Rigid Reservoirs

(71) Applicants: Jason Shih, Yorba Linda, CA (US); Yu-Chong Tai, Pasadena, CA (US); Andrew Dunn, Santa Monica, CA (US)

(72) Inventors: Jason Shih, Yorba Linda, CA (US); Yu-Chong Tai, Pasadena, CA (US); Andrew Dunn, Santa Monica, CA (US)

(21) Appl. No.: 14/984,685

(22) Filed: Dec. 30, 2015

Related U.S. Application Data
(60) Provisional application No. 62/098,742, filed on Dec. 31, 2014.

(51) Int. Cl. A61M 5/145; A61M 5/168; A61M 5/142

(52) U.S. Cl. CPC A61M 5/14593; A61M 5/14276; A61M 5/16804; A61M 2005/14204

(57) ABSTRACT

In various embodiments, an implantable drug-delivery device features one or more rigid drug reservoirs from which therapeutic agents are administered and which are refilled from a flexible reservoir within the device.

### RELATED APPLICATION

This application claims the benefit of and priority to U.S. Provisional Patent Application No. 62/098,742, filed Dec. 31, 2014, the entire disclosure of which is hereby incorporated herein by reference.

### TECHNICAL FIELD

In various embodiments, the present invention relates generally to implantable pumps for, e.g., drug administration, featuring rigid and flexible reservoirs.

### BACKGROUND

Medical treatment often requires the administration of a therapeutic agent (e.g., medicament, drugs, etc.) to a particular part of a patient's body. As patients live longer and are diagnosed with chronic and/or debilitating ailments, the need to place even more protein therapeutics, small-molecule drugs, and other medications into targeted anatomical areas will only increase. Some maladies, however, are difficult to treat with currently available therapies and/or require administration of drugs to difficult-to-reach anatomical regions. Many of these therapies would benefit from concentrated target-area treatment, which would reduce systemic side effects. Furthermore, certain drugs such as protein therapeutics are expensive, costing thousands of dollar per vial. For these reasons, new and improved approaches to targeted drug delivery are constantly sought.

Implantable drug-delivery devices with refillable drug reservoirs address and overcome many of the problems associated with conventional drug-delivery modalities. They generally facilitate controlled delivery of pharmaceutical solutions to a specified target. As the contents of the drug reservoir deplete, a clinician may refill the reservoir in situ, i.e., while leaving the device implanted within the patient's body.

As implantable devices of varying sizes, dosing requirements, and implant locations become available, the reliable delivery of drugs over long periods of time (i.e. bi-weekly or monthly dosing over two or more years) becomes progressively complicated. The functionality of many electrolysis-based pumps is often limited in the fact that for temporally separated doses, each successive dose requires more power to complete. Also, electrolysis-based pumps often have difficulty replicating dose volumes with the accuracy of microliters (μL) and picoliters (pL).

Accordingly, there is a need for implantable pumps that facilitate the delivery of repeatable doses of therapeutic agents.

### SUMMARY

In various embodiments, the present invention relates to drug-delivery pumps that incorporate one or more rigid reservoirs sized to contain a pre-determined amount (e.g., one dose) of a therapeutic agent such as a drug. Actuation of the pump administers this set amount of fluid to a patient, and then the rigid reservoir is refilled from a flexible reservoir containing a much larger volume of the fluid to be administered. In this manner, the administration of set amounts of fluid over time is accomplished via application of the same power level for successive doses. Various embodiments of the invention feature two or more series- and/or parallel-connected rigid reservoirs and pumping chambers (e.g., electrolytic pumping chambers) associated therewith to thereby enable the administration of larger doses and/or multiple doses closely separated in time.

In an aspect, embodiments of the invention feature an implantable drug-delivery device that includes or consists essentially of a rigid housing having an interior, a cannula, a refill port, circuitry, and, disposed within the interior of the housing, (i) a flexible reservoir for containing a therapeutic agent therein, (ii) a rigid envelope, (iii) a diaphragm, and (iv) a check valve. The rigid envelope defines therewithin a rigid reservoir and an expandable electrolysis chamber. The electrolysis chamber contains therewithin a plurality of electrolysis electrodes and an electrolysis fluid. The diaphragm separates the electrolysis chamber from the rigid reservoir. The check valve (i) fluidically connects the flexible reservoir and the rigid reservoir and (ii) is configured to allow flow of liquid from the flexible reservoir to the rigid reservoir but to prevent flow of liquid from the rigid reservoir to the flexible reservoir. The cannula is fluidically coupled to the rigid reservoir and has an exit port outside of the housing. The refill port is fluidically coupled to the flexible reservoir and has an entry port outside of the housing. The circuitry operates the electrodes to (i) cause evolution of gas from the electrolysis fluid to thereby expand the electrolysis chamber within the rigid envelope and drive therapeutic agent from the rigid reservoir out through the cannula and (ii) stop evolution of gas from the electrolysis fluid, resulting dissolution of gas back into the electrolysis fluid generating a vacuum within the rigid reservoir sufficient to crack the check valve and at least partially refill the rigid reservoir with therapeutic agent from the flexible reservoir.

Embodiments of the invention may include one or more of the following in any of a variety of combinations. The check valve may be disposed within the rigid envelope. The diaphragm may be corrugated and/or flexible. The rigid housing may define one or more perforations therethrough. A flow sensor may be disposed between the rigid reservoir and the exit port. A second check valve (i) may fluidically connect the rigid reservoir and the exit port and (ii) may be configured to allow flow of liquid from the rigid reservoir to the exit port but prevent flow of liquid from the exit port to the rigid reservoir.

In another aspect, embodiments of the invention feature an implantable drug-delivery device that includes or consists essentially of a rigid housing having an interior, a cannula having an exit port outside of the housing, a refill port having an entry port outside of the housing, first circuitry, second circuitry, and, disposed within the interior of the housing, (i) a flexible reservoir for containing a therapeutic agent therein, (ii) a first rigid reservoir, (iii) a first check valve, (iv) a first expandable electrolysis chamber, (v) a second rigid reservoir fluidically coupled to the cannula, (vi) a second check valve, and (vii) a second expandable electrolysis chamber. The flexible reservoir is fluidically coupled to the refill port. The first check valve (i) fluidically connects the flexible reservoir and the first rigid reservoir and (ii) is configured to allow flow of liquid from the flexible reservoir to the first rigid reservoir but to prevent flow of liquid from the first rigid reservoir to the flexible reservoir. The first expandable electrolysis chamber contains therewithin a plurality of electrolysis electrodes and an electrolysis fluid. The second check valve (i) fluidically connects the first rigid reservoir and the second rigid reservoir and (ii) is configured to allow flow of liquid from the first rigid reservoir to the second rigid reservoir but to prevent flow of liquid from the second rigid reservoir to the first rigid reservoir. The second expandable electrolysis chamber contains therewithin a plurality of electrolysis electrodes and an electrolysis fluid. The first circuitry operates the electrodes of the first electrolysis chamber to cause evolution of gas from the electrolysis fluid in the first electrolysis chamber to thereby expand the first electrolysis chamber and drive therapeutic agent from the first rigid reservoir into the second rigid reservoir. When the therapeutic agent from the first rigid reservoir is driven into the second rigid reservoir, the therapeutic agent may fill at least a portion of the second rigid reservoir, particularly if the second rigid reservoir is initially empty or only partially filled. If the second rigid reservoir contains or is substantially filled with therapeutic agent, then driving additional therapeutic agent into the second rigid reservoir from the first rigid reservoir drives a volume of the collective volume of therapeutic agent (i.e., the collective volume of the therapeutic agent already present in the second rigid reservoir and the therapeutic agent being driven in from the first rigid reservoir) exceeding the volume of the second rigid reservoir is driven out through the cannula. The first circuitry also operates the electrodes of the first electrolysis chamber to stop evolution of gas from the electrolysis fluid in the first electrolysis chamber, resulting dissolution of gas back into the electrolysis fluid in the first electrolysis chamber generating a vacuum within the first rigid reservoir sufficient to crack the first check valve and at least partially refill the first rigid reservoir with therapeutic agent from the flexible reservoir. The second circuitry operates the electrodes of the second electrolysis chamber to (i) cause evolution of gas from the electrolysis fluid in the second electrolysis chamber to thereby expand the second electrolysis chamber and drive therapeutic agent from the second rigid reservoir out through the cannula and (ii) stop evolution of gas from the electrolysis fluid in the second electrolysis chamber, resulting dissolution of gas back into the electrolysis fluid in the second electrolysis chamber generating a vacuum within the second rigid reservoir sufficient to crack the second check valve and at least partially refill the second rigid reservoir with therapeutic agent from the first rigid reservoir.

In yet another aspect, embodiments of the invention feature an implantable drug-delivery device that includes or consists essentially of a rigid housing having an interior, a cannula having an exit port outside of the housing, a refill port having an entry port outside of the housing, first circuitry, second circuitry, and, disposed within the interior of the housing, (i) a flexible reservoir for containing a therapeutic agent therein, (ii) a first rigid reservoir fluidically coupled to the cannula, (iii) a first check valve, (iv) a first expandable electrolysis chamber, (v) a second rigid reservoir fluidically coupled to the cannula, (vi) a second check valve, and (vii) a second expandable electrolysis chamber. The flexible reservoir is fluidically coupled to the refill port. The first check valve (i) fluidically connects the flexible reservoir and the first rigid reservoir and (ii) is configured to allow flow of liquid from the flexible reservoir to the first rigid reservoir but to prevent flow of liquid from the first rigid reservoir to the flexible reservoir. The first expandable electrolysis chamber contains therewithin a plurality of electrolysis electrodes and an electrolysis fluid. The second check valve (i) fluidically connects the flexible reservoir and the second rigid reservoir and (ii) is configured to allow flow of liquid from the flexible reservoir to the second rigid reservoir but to prevent flow of liquid from the second rigid reservoir to the flexible reservoir. The second expandable electrolysis chamber contains therewithin a plurality of electrolysis electrodes and an electrolysis fluid. The first circuitry operates the electrodes of the first electrolysis chamber to (i) cause evolution of gas from the electrolysis fluid in the first electrolysis chamber to thereby expand the first electrolysis chamber and drive therapeutic agent from the first rigid reservoir out through the cannula and (ii) stop evolution of gas from the electrolysis fluid in the first electrolysis chamber, resulting dissolution of gas back into the electrolysis fluid in the first electrolysis chamber generating a vacuum within the first rigid reservoir sufficient to crack the first check valve and at least partially refill the first rigid reservoir with therapeutic agent from the flexible reservoir. The second circuitry operates the electrodes of the second electrolysis chamber to (i) cause evolution of gas from the electrolysis fluid in the second electrolysis chamber to thereby expand the second electrolysis chamber and drive therapeutic agent from the second rigid reservoir out through the cannula and (ii) stop evolution of gas from the electrolysis fluid in the second electrolysis chamber, resulting dissolution of gas back into the electrolysis fluid in the second electrolysis chamber generating a vacuum within the second rigid reservoir sufficient to crack the second check valve and at least partially refill the second rigid reservoir with therapeutic agent from the flexible reservoir.

In another aspect, embodiments of the invention feature a method for administering a therapeutic agent from an implantable drug-delivery device. In a step (a), a dose of the therapeutic agent is urged from a rigid reservoir, through a cannula, and out an exit port of the cannula. In a step (b) after step (a), a vacuum is generated within the rigid reservoir, the vacuum inducing flow of therapeutic agent into the rigid reservoir from a flexible reservoir to replace the dose urged from the rigid reservoir.

Embodiments of the invention may include one or more of the following in any of a variety of combinations. Steps (a) and (b) may be repeated one or more times without refilling the flexible reservoir with therapeutic agent. Urging the dose of therapeutic agent from the rigid reservoir may include, consist essentially of, or consist of electrolyzing a fluid to generate gas therefrom, thereby applying a positive pressure on an interior of the rigid reservoir. Generating the vacuum within the rigid reservoir may include, consist essentially of, or consist of dissolving gas back into the fluid (via, e.g., halting the electrolysis process), thereby applying a negative pressure on the interior of the rigid reservoir. Steps (a) and (b) may be repeated one or more times, without refilling the flexible reservoir with therapeutic agent, to urge one or more doses of the therapeutic agent from the rigid reservoir. Electrolyzing the fluid may include, consist essentially of, or consist of applying power to electrolysis electrodes. The power applied to the electrolysis electrodes for each dose may be substantially constant notwithstanding a diminishing volume of therapeutic agent disposed within the flexible reservoir. The vacuum generated within the rigid reservoir may crack a check valve disposed between the flexible reservoir and the rigid reservoir.

In yet another aspect, embodiments of the invention feature an implantable drug-delivery device that includes or consists essentially of a rigid housing having an interior, a cannula having an exit port outside of the housing, a refill port having an entry port outside of the housing, circuitry, and, disposed within the interior of the housing, (i) a flexible reservoir for containing a therapeutic agent therein, (ii) a rigid envelope, (iii) a diaphragm, and (iv) a check valve. The rigid envelope defines therewithin (i) a rigid reservoir and (ii) a pressure-actuated pump chamber containing therewithin a reversible pressure-actuation mechanism. The diaphragm separates the pressure-actuated pump chamber from the rigid reservoir. The check valve (i) fluidically connects the flexible reservoir and the rigid reservoir and (ii) is configured to allow flow of liquid from the flexible reservoir to the rigid reservoir but to prevent flow of liquid from the rigid reservoir to the flexible reservoir. The cannula is fluidically coupled to the rigid reservoir. The refill port is fluidically coupled to the flexible reservoir. The circuitry operates the pressure-actuation mechanism to (i) cause an increase in pressure in the pump chamber to thereby expand the pump chamber and drive therapeutic agent from the rigid reservoir out through the cannula and (ii) cause a decrease in pressure in the pump chamber to thereby generate a vacuum within the rigid reservoir sufficient to crack the check valve and at least partially refill the rigid reservoir with therapeutic agent from the flexible reservoir. The pressure-actuation mechanism may include, consist essentially of, or consists of a heating mechanism and a phase-change material (e.g., an ambient temperature-range phase-change material).

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “approximately” and “substantially” mean ±10%, and in some embodiments, ±5%. The term “consists essentially of” means excluding other materials that contribute to function, unless otherwise defined herein. Nonetheless, such other materials may be present, collectively or individually, in trace amounts.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic sectional view of a drug-delivery pump implanted at a drug administration site (e.g., in a patient's eye) in accordance with various embodiments of the invention; and

FIGS. 2 and 3 are schematic block diagrams of drug-delivery pumps featuring multiple rigid reservoirs in accordance with various embodiments of the invention.

### DETAILED DESCRIPTION

Embodiments of the present invention relate, generally, to implantable drug pump devices with refillable drug reservoirs. Various embodiments described herein relate specifically to drug pump devices implanted into the eye (e.g., between the sclera and conjunctiva); however, many features relevant to such ophthalmic pumps are also applicable to other drug pump devices, such as, e.g., implantable insulin pumps, inner ear pumps, and brain pumps.

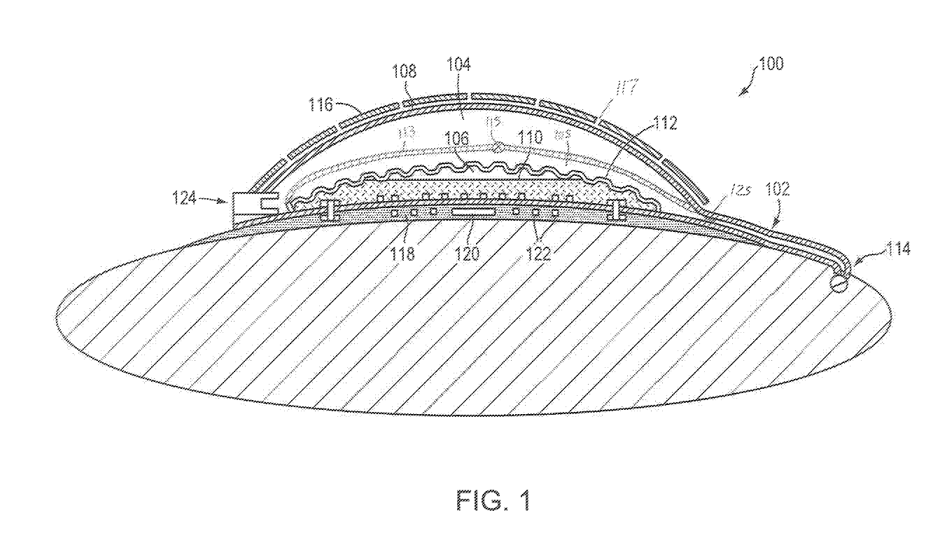

FIG. 1 illustrates an exemplary electrolytically driven drug pump device 100 in accordance herewith (aspects of which are described in detail in U.S. application Ser. Nos. 12/463,251 and 13/632,644, the entire disclosures of which are hereby incorporated by reference). The drug pump device 100 includes a cannula 102 and three chambers 104, 105, 106 disposed within a flexible envelope 108. The flexible envelope 108 may at least partially enclose top chamber 104, and it may include or consist essentially of, e.g., a flexible material such as an elastomer. The top chamber (or “flexible reservoir”) 104 defines a drug reservoir that contains the drug to be administered in liquid form, the middle chamber (or “rigid reservoir”) 105 is a reservoir fluidly connected to the cannula 102 and sized to contain a pre-determined amount of the drug received from the flexible reservoir 104, and the bottom chamber (or “pumping chamber”) 106 contains a liquid which, when subjected to electrolysis using electrolysis electrodes 110, evolves a gaseous product. The electrolyte liquid contained within the pumping chamber 106 may include, consist essentially of, or consist of, for example, a saline (i.e., NaCl and H₂O) solution, a solution that contains either magnesium sulfate or sodium sulfate, pure water, or any non-toxic solution. The chambers 105, 106 are separated by a diaphragm 112. The diaphragm 112 may be elastic and/or may be corrugated to provide for expansion thereof in response to the phase-change of the fluid within the bottom chamber 106 from a liquid to a gaseous state. The diaphragm 112 may be manufactured from, for example, one or more parylene films and/or a composite material. Various embodiments of the invention feature pumping chambers 106 shaped to conform to the movement of diaphragms or other actuation members having shapes other than the curved corrugated shape depicted in FIG. 1, e.g., the actuation member may be a piston or bellows deflecting in only one dimension.

The chambers 104, 105 are separated by a rigid envelope 113 that maintains its shape (and therefore the collective volume of chambers 105, 106) regardless of the amount of the drug present therewithin or within flexible reservoir 104. The rigid envelope 113 may include or consist essentially of one or more substantially rigid materials that are biocompatible (e.g., medical-grade polypropylene and/or a biocompatible plastic), and it does not flex or deform during operation of pumping chamber 106 or as a function of the amount of drug contained within chamber 104. (Note that, as utilized herein, the term “flexible reservoir” does not require that the entire outer boundary of the reservoir is flexible, only that at least a portion of the boundary is flexible and therefore capable of expanding and/or deforming in response to different amounts of fluid contained therein. Similarly, the term “rigid reservoir” does not require that the entire outer boundary of the reservoir is rigid, only that the reservoir does not expand in size in response to force applied by a pumping chamber; a portion of the outer boundary of a rigid reservoir may be defined by the diaphragm or other actuation member of the pumping chamber.) The cannula 102 connects the rigid reservoir 105 with a check valve 114 inserted at the site of administration or anywhere along the fluid path between the rigid reservoir and site of administration. The check valve 114 is configured to allow flow of liquid out of the cannula 102 to the administration size but not to allow flow of liquid back from the administration site into the cannula 102 and/or into the rigid reservoir 105. The rigid envelope 113 separating chambers 104, 105 features thereon or therewithin a check valve 115 that enables flow of the drug from flexible reservoir 104 into the rigid reservoir 105 without allowing flow in the opposite direction. The flexible envelope 108 resides within a shaped protective shell 116 typically made of a relatively rigid biocompatible material (e.g., medical-grade polypropylene). As shown, the shell 116 may define one or more perforations 117 therewithin; such perforations advantageously prevent the formation of a vacuum between the flexible envelope 108 and the shell 116 upon deformation of the flexible envelope 108 toward the rigid envelope 113. Alternatively, a separate bladder fluidically connected to the region between the flexible envelope and shell may interface with the external environment to prevent formation of a vacuum.

Control circuitry 118, a battery 120, and an induction coil 122 for power and data transmission are embedded between the bottom wall of the electrolyte chamber 106 and the floor of the shell 116. Depending on the complexity of the control functionality it provides, the control circuitry 118 may be implemented, e.g., in the form of analog circuits, digital integrated circuits (such as, e.g., microcontrollers), or programmable logic devices. In some embodiments, the control circuitry 118 controls the pumping action of pump 100 and includes a microprocessor and associated memory for implementing complex drug-delivery protocols. The drug pump device 100 may also include various sensors (e.g., pressure and flow sensors) for monitoring the status and operation of the various device components, and such data may be logged in the memory for subsequent retrieval and review. In various embodiments, the induction coil 122 permits wireless (e.g., radio-frequency (RF)) communication with an external controller (e.g., a portable control handset), which may also be used, for example, to charge the battery 120. The coil 122 may be or resemble, for example, a coil described in U.S. patent application Ser. No. 13/491,741, filed on Jun. 8, 2012, the entire disclosure of which is incorporated by reference herein. The external controller may be used to send wireless signals to the control circuitry 118 in order to program, reprogram, operate, calibrate, or otherwise configure the operation of the pump 100. The control circuitry 118 may, for example, communicate electrically with the electrolysis electrodes 110 by means of metal interconnects extending thereto.

Implantable, refillable drug pump devices need not, of course, have the particular configuration depicted in FIG. 1. Various modifications are possible, including, e.g., devices in which the flexible reservoir, rigid reservoir, and pump chamber are arranged side-by-side (rather than one above the other), and/or in which pressure generated in the pump chamber is exerted on the rigid reservoir via a piston (rather than by a flexible diaphragm). Furthermore, the pump need not in all embodiments be driven electrolytically, but may exploit, e.g., osmotic or electroosmotic drive mechanisms, mechanisms utilizing high vapor-pressure propellant, phase-change materials operative at ambient temperature, reactive materials triggerable by catalyst introduction, or even pressure generated manually. In various embodiments, the pressure-actuation mechanism is reversible and therefore capable of exerting both positive and negative pressure on the rigid reservoir (i.e., capable of both increasing and decreasing the pressure in the pump chamber). In embodiments using a phase-change material, a heating mechanism (e.g., a resistive heater) may be used to alter the temperature within the pump chamber and thereby alter the phase of the material to selectively increase and decrease pump chamber pressure; in such embodiments, the pressure-actuation mechanism may include or consist essentially of both the heating mechanism and the phase-change material. Phase-change materials used in accordance with embodiments of the invention may have transformation temperatures (e.g., melting points) within, e.g., approximately ±10° C. of ambient (e.g., room) temperature and/or of the body temperature of a patient (e.g., a human patient) in which drug pump device 100 may be implanted. Transformation temperatures of phase-change materials used in accordance with embodiments of the invention may range from, for example, approximately 16° C. to approximately 45° C.

Importantly for the prolonged use of the drug pump device 100 following implantation, the device 100 includes one or more refill ports 124 in fluid communication at least with the flexible reservoir 104, which permit a refill needle (not shown) to be inserted therethrough. Each refill port 124 may have a venting arrangement integrated therewith for, e.g., the venting of excess gas and/or pressure equalization, as described in U.S. patent application Ser. No. 14/317,848, filed Jun. 27, 2014, or U.S. patent application Ser. No. 14/807,940, filed Jul. 24, 2015, the entire disclosure of each of which is hereby incorporated by reference herein. As shown in FIG. 1, in embodiments of the present invention, the flexible reservoir 104 is not directly fluidically connected to the cannula 102; rather, liquid from the flexible reservoir 104 is utilized to fill one or more rigid reservoirs that themselves are fluidically connected to the cannula 102. Moreover, in embodiments of the present invention, the rigid reservoir(s) are not directly fluidically connected to the refill port 124; rather, refilling of device 100 involves introduction of liquid into the flexible reservoir 104 via the refill port 124, and the liquid is introduced into the rigid reservoir(s) from the flexible reservoir 104.

The presence of rigid reservoir 105 within the drug pump device advantageously enables the administration of a pre-determined volume, i.e., the volume of the rigid reservoir 105, or fraction thereof, during each actuation cycle. In an exemplary embodiment, the rigid reservoir 105 contains a volume of (and may even be substantially filled with) the drug from the flexible reservoir 104. When current is supplied to the electrolysis electrodes 110 by the circuitry 118, the electrolytic fluid within the bottom chamber 106 evolves into a gas. This phase change increases the volume of the bottom chamber 106, thereby expanding the diaphragm 112 and forcing liquid out of the rigid reservoir 105, through the cannula 102, and toward the treatment site. In various embodiments, the volume of rigid reservoir 105 is approximately equal to the dose volume (and may be slightly larger to accommodate downstream fluid loss); in such cases, actuation of the diaphragm 112 fully empties the rigid reservoir 105, thereby removing the requirement for continuous feedback monitoring to deliver accurate doses that are additive or multiple sums of the one or more rigid reservoir volumes. Alternatively or as an added safety measure, a flow sensor 125 disposed proximate or within the cannula 102 may measure the outflow of the drug from the rigid reservoir 105 and may signal (via circuitry 118) the end of an actuation cycle once the pre-determined volume of the drug has been administered.

When current to the electrolysis electrodes 110 is stopped, the gas within the bottom chamber 106 dissipates back into its liquid state, and the diaphragm 112 of the electrolysis chamber relaxes back to its original configuration. The relaxation of the diaphragm 112 generates a vacuum in the rigid reservoir 105 because envelope 113 is substantially rigid. The vacuum generated by the relaxation of the diaphragm 112 cracks the check valve 115, enabling flow of the drug from the flexible reservoir 104 to refill the rigid reservoir 105. (In addition, the presence of check valve 114 prevents the vacuum generated by the relaxation of the diaphragm 112 from resulting in ingress of fluid from the administration site through the cannula 102.) Thus, once dissociation within the chamber 106 and concomitant relaxation of diaphragm 112 are complete, the rigid reservoir 105 is again filled with the drug and the volume of flexible reservoir 104 has decreased by the volume of fluid ejected from the rigid reservoir 105 (e.g., one dose of the drug). This process may be repeated for administration of successive doses of the drug, and, when necessary, the flexible reservoir 104 may be refilled via refill port 124. If the rigid reservoir 105 has a defined, unchanging volume that is fully emptied upon actuation of the diaphragm 112 and is refilled with the same volume of the drug for each dose administration, the amount of power required to deliver each dose is substantially identical. In contrast, the amount of power required to deliver successive doses from a flexible drug reservoir will tend to vary as a function of the remaining drug reservoir volume and the pressure actuation mechanism's (e.g., the diaphragm's) required deflection for pressure transmission.

In various embodiments, the flow sensor 125 may even be omitted from drug delivery pump 100. If the rigid reservoir 105 is sized to contain the desired dose volume, then the pump may be programmed to simply deliver the entire volume of rigid reservoir 105 during each actuation. If the volume of the rigid reservoir 105 is approximately equal to or less than the prescribed dose, then embodiments of the invention advantageously prevent overdosing of the drug, as a single actuation of the drug pump 100 will be incapable of administering an overdose.

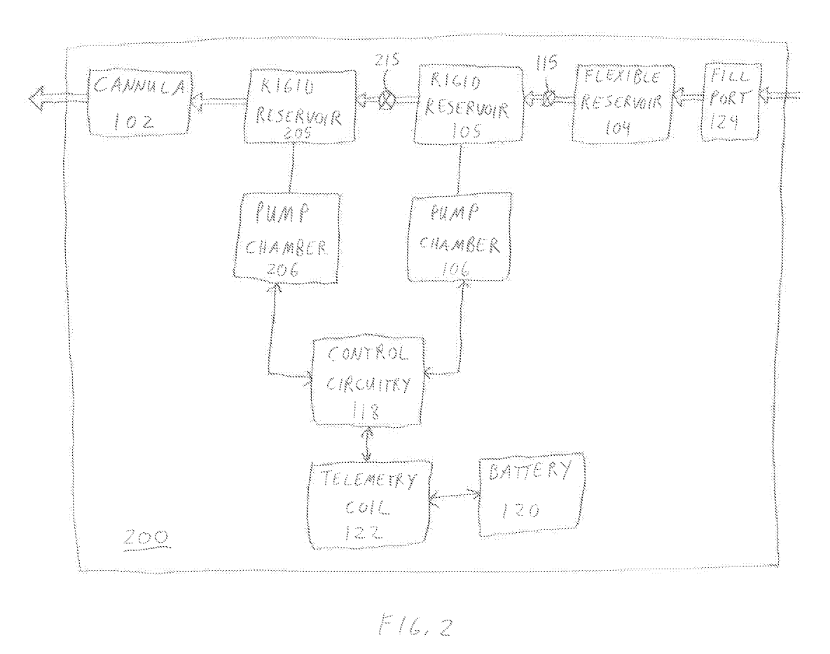

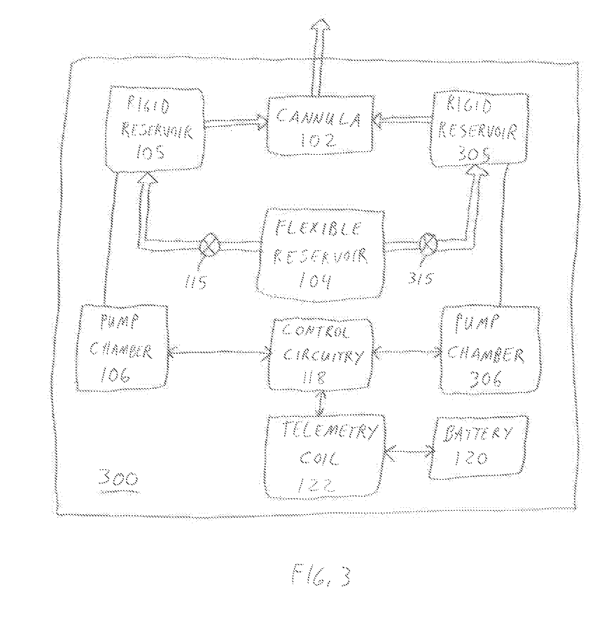

As shown schematically in FIG. 2, embodiments of the invention feature drug delivery pumps 200 having multiple rigid reservoirs (and associated pumping chambers). (In FIGS. 2 and 3, fluid flow is represented by open arrows, and solid arrows signify the flow of electronic signals or communications.) Specifically, pump 200 features two series-connected rigid reservoirs 105, 205 with associated pump chambers 106, 206 that actuate to draw fluid (e.g., a drug) from the flexible reservoir 104. The multiple rigid reservoirs of pump 200 enable redundancy in the event of failure of one of the pump chambers 106, 206. The rigid reservoirs 102, 205 are separated by a check valve 215 that enables fluid flow from rigid reservoir 105 to rigid reservoir 205 but not in the opposite direction. Only one of the pump chambers 106, 206 need be active for dosing at a time, and the rigid reservoir associated with the inactive pump chamber merely functions as a “pass-through” for the drug to be administered. For example, if pump chamber 106 is actively actuating, relaxation of the diaphragm therein generates a vacuum within the rigid reservoir 105, drawing fluid into rigid reservoir 105 from the flexible reservoir 104. Rigid reservoir 205 will typically already be substantially filled with the drug, or pump chamber 106 can actuate one or more cycles until drug from rigid reservoir 105 fills the rigid reservoir 205. Then, upon actuation of pump chamber 106, the drug from rigid reservoir 105 is forced into rigid reservoir 205, and drug therein flows through the cannula 102 to the administration site.

Similarly, if pump chamber 206 is actively actuating, relaxation of the diaphragm therein generates a vacuum within the rigid reservoir 205, drawing fluid into rigid reservoir 205 from the rigid reservoir 105. The accompanying vacuum generated in rigid reservoir 105 serves to draw additional fluid into rigid reservoir 105 from the flexible reservoir 104, refilling rigid reservoir 105. Then, upon actuation of pump chamber 206, the drug from rigid reservoir 205 flows through the cannula 102 to the administration site.

The pump 200 featuring multiple rigid reservoirs and pump actuation chambers may also be utilized to administer two doses in quick succession. Conventional electrolytic drug pumps may be unable to administer multiple doses in a short period of time due to the need for electrolytic recovery (i.e., gas dissolution back into liquid) processes that may last minutes or even hours, depending upon the size of the electrolytic pumping chamber. Thus, pump 200 may be utilized to administer a “rescue dose” if required by a patient, or the volumes of the rigid reservoirs 105, 205 may be different from each other, enabling the administration of a variety of different doses from one reservoir, the other reservoir, or both reservoirs.

In an exemplary embodiment, pump chamber 106 may be actuated, thereby forcing the drug from rigid reservoir 105 through rigid reservoir 205 into the cannula 102. Relaxation of the diaphragm of pump chamber 106 generates a vacuum within the rigid reservoir 105, drawing fluid into rigid reservoir 105 from the flexible reservoir 104. While rigid reservoir 105 is being refilled, the pump chamber 206 may be actuated, thereby forcing the drug from rigid reservoir 205 into the cannula 102. Subsequent relaxation of the diaphragm of pump chamber 206 generates a vacuum within the rigid reservoir 206, drawing fluid into rigid reservoir 205 from rigid reservoir 105; the resulting vacuum in rigid reservoir 105 draws more fluid into rigid reservoir 105 from the flexible reservoir 104. In this manner, larger doses (than a volume of one of the rigid reservoirs) may be administered, and/or more frequent dosing is enabled, as a dose may be administered during recovery of one of the pump chambers.

Embodiments of the invention may incorporate multiple rigid reservoirs and associated pump chambers in any of a variety of series- and/or parallel-connected configurations. For example, FIG. 3 depicts a pump 300 that features two parallel-connected rigid reservoirs 105, 305 with associated pump chambers 106, 306 that actuate to draw fluid (e.g., a drug) from the flexible reservoir 104. (The fill port 124 is not shown in FIG. 3 for clarity.) As in pump 100 of FIG. 1, check valve 115 separates flexible reservoir 104 from rigid reservoir 105 and allows fluid flow only from flexible reservoir 104 into rigid reservoir 105; a check valve 315 separates flexible reservoir 104 from rigid reservoir 305 and allows fluid flow only from flexible reservoir 104 into rigid reservoir 305. As described above for pump 200, the presence of multiple rigid reservoirs and associated pumping chambers may provide redundancy in the event of failure of one of the pumping chambers; in such an event, the remaining pumping chamber and rigid reservoir may be utilized to administer specific doses of the drug from flexible reservoir 104 as described above for pump 100. The parallel rigid reservoirs 105, 305 may also be utilized to administer multiple doses in quick succession or larger doses than that enabled by the volume of one of the rigid reservoirs, as described above for pump 200. In pump 300, one pump chamber may be actuated to administer the drug residing in its associated rigid reservoir, and while that rigid reservoir is being refilled from flexible reservoir 104 during pump-chamber recovery, the other pump chamber may be actuated to administer the drug from its associated rigid reservoir. In this manner, the drug may be administered from one rigid reservoir during refilling of the other, and vice versa. The drug administration may be programmed to mimic that of a basal rate, bolus, or a combination thereof.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An implantable drug-delivery device comprising:
a rigid housing having an interior;
disposed within the interior of the housing:
a flexible reservoir for containing a therapeutic agent therein,
a rigid envelope defining therewithin (i) a rigid reservoir and (ii) an expandable electrolysis chamber comprising therewithin a plurality of electrolysis electrodes and an electrolysis fluid,
a diaphragm separating the electrolysis chamber from the rigid reservoir; and
a check valve (i) fluidically connecting the flexible reservoir and the rigid reservoir and (ii) configured to allow flow of liquid from the flexible reservoir to the rigid reservoir but to prevent flow of liquid from the rigid reservoir to the flexible reservoir;
a cannula fluidically coupled to the rigid reservoir and having an exit port outside of the housing;
a refill port fluidically coupled to the flexible reservoir and having an entry port outside of the housing; and
circuitry for operating the electrodes to (i) cause evolution of gas from the electrolysis fluid to thereby expand the electrolysis chamber within the rigid envelope and drive therapeutic agent from the rigid reservoir out through the cannula and (ii) stop evolution of gas from the electrolysis fluid, resulting dissolution of gas back into the electrolysis fluid generating a vacuum within the rigid reservoir sufficient to crack the check valve and at least partially refill the rigid reservoir with therapeutic agent from the flexible reservoir.

2. The device of claim 1, wherein the check valve is disposed within the rigid envelope.

3. The device of claim 1, wherein the diaphragm is corrugated.

4. The device of claim 1, wherein the diaphragm is flexible.

5. The device of claim 1, wherein the rigid housing defines one or more perforations therethrough.

6. The device of claim 1, further comprising a flow sensor disposed between the rigid reservoir and the exit port.

7. The device of claim 1, further comprising a second check valve (i) fluidically connecting the rigid reservoir and the exit port and (ii) configured to allow flow of liquid from the rigid reservoir to the exit port but prevent flow of liquid from the exit port to the rigid reservoir.

8. An implantable drug-delivery device comprising:
a rigid housing having an interior;
a cannula having an exit port outside of the housing;
a refill port having an entry port outside of the housing;
disposed within the interior of the housing:
fluidically coupled to the refill port, a flexible reservoir for containing a therapeutic agent therein,
a first rigid reservoir,
a first check valve (i) fluidically connecting the flexible reservoir and the first rigid reservoir and (ii) configured to allow flow of liquid from the flexible reservoir to the first rigid reservoir but to prevent flow of liquid from the first rigid reservoir to the flexible reservoir,
a first expandable electrolysis chamber comprising therewithin a plurality of electrolysis electrodes and an electrolysis fluid,
a second rigid reservoir fluidically coupled to the cannula,
a second check valve (i) fluidically connecting the first rigid reservoir and the second rigid reservoir and (ii) configured to allow flow of liquid from the first rigid reservoir to the second rigid reservoir but to prevent flow of liquid from the second rigid reservoir to the first rigid reservoir, and
a second expandable electrolysis chamber comprising therewithin a plurality of electrolysis electrodes and an electrolysis fluid;
circuitry for operating the electrodes of the first electrolysis chamber to (i) cause evolution of gas from the electrolysis fluid in the first electrolysis chamber to thereby expand the first electrolysis chamber and drive therapeutic agent from the first rigid reservoir into the second rigid reservoir and (ii) stop evolution of gas from the electrolysis fluid in the first electrolysis chamber, resulting dissolution of gas back into the electrolysis fluid in the first electrolysis chamber generating a vacuum within the first rigid reservoir sufficient to crack the first check valve and at least partially refill the first rigid reservoir with therapeutic agent from the flexible reservoir; and
circuitry for operating the electrodes of the second electrolysis chamber to (i) cause evolution of gas from the electrolysis fluid in the second electrolysis chamber to thereby expand the second electrolysis chamber and drive therapeutic agent from the second rigid reservoir out through the cannula and (ii) stop evolution of gas from the electrolysis fluid in the second electrolysis chamber, resulting dissolution of gas back into the electrolysis fluid in the second electrolysis chamber generating a vacuum within the second rigid reservoir sufficient to crack the second check valve and at least partially refill the second rigid reservoir with therapeutic agent from the first rigid reservoir.

9. An implantable drug-delivery device comprising:
a rigid housing having an interior;
a cannula having an exit port outside of the housing;
a refill port having an entry port outside of the housing;
disposed within the interior of the housing:
fluidically coupled to the refill port, a flexible reservoir for containing a therapeutic agent therein,
a first rigid reservoir fluidically coupled to the cannula,
a first check valve (i) fluidically connecting the flexible reservoir and the first rigid reservoir and (ii) configured to allow flow of liquid from the flexible reservoir to the first rigid reservoir but to prevent flow of liquid from the first rigid reservoir to the flexible reservoir,
a first expandable electrolysis chamber comprising therewithin a plurality of electrolysis electrodes and an electrolysis fluid,
a second rigid reservoir fluidically coupled to the cannula,
a second check valve (i) fluidically connecting the flexible reservoir and the second rigid reservoir and (ii) configured to allow flow of liquid from the flexible reservoir to the second rigid reservoir but to prevent flow of liquid from the second rigid reservoir to the flexible reservoir,
a second expandable electrolysis chamber comprising therewithin a plurality of electrolysis electrodes and an electrolysis fluid;
circuitry for operating the electrodes of the first electrolysis chamber to (i) cause evolution of gas from the electrolysis fluid in the first electrolysis chamber to thereby expand the first electrolysis chamber and drive therapeutic agent from the first rigid reservoir out through the cannula and (ii) stop evolution of gas from the electrolysis fluid in the first electrolysis chamber, resulting dissolution of gas back into the electrolysis fluid in the first electrolysis chamber generating a vacuum within the first rigid reservoir sufficient to crack the first check valve and at least partially refill the first rigid reservoir with therapeutic agent from the flexible reservoir; and
circuitry for operating the electrodes of the second electrolysis chamber to (i) cause evolution of gas from the electrolysis fluid in the second electrolysis chamber to thereby expand the second electrolysis chamber and drive therapeutic agent from the second rigid reservoir out through the cannula and (ii) stop evolution of gas from the electrolysis fluid in the second electrolysis chamber, resulting dissolution of gas back into the electrolysis fluid in the second electrolysis chamber generating a vacuum within the second rigid reservoir sufficient to crack the second check valve and at least partially refill the second rigid reservoir with therapeutic agent from the flexible reservoir.

10. A method for administering a therapeutic agent from an implantable drug-delivery device, the method comprising:
(a) urging a dose of the therapeutic agent from a rigid reservoir, through a cannula, and out an exit port of the cannula; and
(b) thereafter, generating a vacuum within the rigid reservoir, the vacuum inducing flow of therapeutic agent into the rigid reservoir from a flexible reservoir to replace the dose urged from the rigid reservoir.

11. The method of claim 10, further comprising repeating steps (a) and (b) one or more times without refilling the flexible reservoir with therapeutic agent.

12. The method of claim 10, wherein urging the dose of therapeutic agent from the rigid reservoir comprises electrolyzing a fluid to generate gas therefrom, thereby applying a positive pressure on an interior of the rigid reservoir.

13. The method of claim 12, wherein generating the vacuum within the rigid reservoir comprises dissolving gas back into the fluid, thereby applying a negative pressure on the interior of the rigid reservoir.

14. The method of claim 12, further comprising repeating steps (a) and (b) one or more times, without refilling the flexible reservoir with therapeutic agent, to urge one or more doses of the therapeutic agent from the rigid reservoir, wherein:
electrolyzing the fluid comprises applying power to electrolysis electrodes, and
the power applied to the electrolysis electrodes for each dose is substantially constant notwithstanding a diminishing volume of therapeutic agent disposed within the flexible reservoir.

15. The method of claim 10, wherein the vacuum generated within the rigid reservoir cracks a check valve disposed between the flexible reservoir and the rigid reservoir.

16. An implantable drug-delivery device comprising:
a rigid housing having an interior;
disposed within the interior of the housing:
a flexible reservoir for containing a therapeutic agent therein,
a rigid envelope defining therewithin (i) a rigid reservoir and (ii) a pressure-actuated pump chamber comprising therewithin a reversible pressure-actuation mechanism,
a diaphragm separating the pressure-actuated pump chamber from the rigid reservoir; and
a check valve (i) fluidically connecting the flexible reservoir and the rigid reservoir and (ii) configured to allow flow of liquid from the flexible reservoir to the rigid reservoir but to prevent flow of liquid from the rigid reservoir to the flexible reservoir;
a cannula fluidically coupled to the rigid reservoir and having an exit port outside of the housing;
a refill port fluidically coupled to the flexible reservoir and having an entry port outside of the housing; and
circuitry for operating the pressure-actuation mechanism to (i) cause an increase in pressure in the pump chamber to thereby expand the pump chamber and drive therapeutic agent from the rigid reservoir out through the cannula and (ii) cause a decrease in pressure in the pump chamber to thereby generate a vacuum within the rigid reservoir sufficient to crack the check valve and at least partially refill the rigid reservoir with therapeutic agent from the flexible reservoir.

17. The device of claim 16, where the pressure-actuation mechanism comprises a heating mechanism and a phase-change material.
